(19) United States
(12) Patent Application Publication
(10) Pub. No.: US 2025/0152415 A1
(43) Pub. Date: May 15, 2025

(54) OCULAR SURGICAL MAGNET APPARATUS

(71) Applicant: EMMETROPE OPHTHALMICS LLC, Menlo Park, CA (US)

(72) Inventors: Jeffrey L. Goldberg, Menlo Park, CA (US); Noelia J. Kunzevitzky, Menlo Park, CA (US)

(21) Appl. No.: 18/839,412

(22) PCT Filed: Feb. 17, 2023
(86) PCT No.: PCT/US2023/062784

§ 371 Date: Aug. 16, 2024

Related U.S. Application Data
(60) Provisional application No. 63/311,712, filed on Feb. 18, 2022.

(51) Int. Cl. A61F 9/007; A61B 17/00; A61B 17/06

(52) U.S. Cl. CPC A61F 9/00709; A61B 2017/00876; A61B 17/06161

(57) ABSTRACT

The present disclosure is directed to an ocular magnetic surgical device. The device includes a magnetic body having an arcuate surface and configured for internal placement behind an eye of a patient. The magnetic body is typically covered by an inert coating. The device also includes an attachment apparatus having an arcuate radius of curvature substantially similar to a radius of curvature of the magnetic body. The attachment apparatus is coupled to the magnetic body having at least one opening disposed along a perimeter of the magnetic body that is configured to receive sutures for coupling the magnetic body adjacent a rear portion of the eye.

### RELATED APPLICATIONS

This application claims priority to and benefit of U.S. provisional patent application 63/311,712, filed Feb. 18, 2022, to Emmetrope Ophthalmics LLC, titled “Ocular Surgical Magnet Apparatus,” the entirety of the disclosure is hereby incorporated herein by reference thereto.

### TECHNICAL FIELD

This document relates to magnetic surgical apparatuses configured to position the magnetic body behind or within the eye of the patient.

### BACKGROUND

Delivery of cells to targeted tissues with the body has therapeutic promise. There is a need in the art for improved processes and apparatuses to assist in such delivery.

### SUMMARY

In some aspects, the disclosure concerns magnetic surgical apparatuses, comprising: a magnetic body comprising an arcuate surface and configured for internal placement behind an eye of a patient, the arcuate surface comprising an area in a range of 1 to 400 mm²; an inert coating disposed around the magnet; an attachment apparatus comprising an arcuate radius of curvature substantially similar to a radius of curvature of the magnetic body, the attachment apparatus coupled to the magnetic body and further comprising at least one opening disposed along a perimeter of the magnetic body, the at least one opening configured to receive sutures for coupling the magnetic body adjacent a rear portion of the eye; an arm with a first end coupled to the magnetic body and a second end of the arm opposite the first end of the arm; and a handle coupled to the second end of the arm, the handle configured to position the magnetic body behind the eye of the patient.

In some embodiments, the arcuate surface comprises a first arcuate line taken along a first direction of the accurate surface, and a second arcuate line taken along a second direction orthogonal to the first arcuate line to form a concave surface configured to mateably couple adjacent a rear surface of the eye.

In certain embodiments the magnetic body comprises arcuate surfaces in orthogonal directions to form a concave surface configured to mateably couple adjacent a rear surface of the eye.

In some magnetic surgical apparatuses, the arcuate radius of curvature of the attachment apparatus is substantially similar to the arcuate radius of curvature of the magnetic body.

In certain magnetic surgical apparatuses, the arm is releasably coupled to the magnetic body and configured to position the magnet behind the eye and then be released from the magnetic body and removed from behind the eye while the magnet remains behind the eye.

In some embodiments, the arm is permanently coupled to the magnetic body and is configured to position the magnet behind the eye and then be removed with the magnetic body from behind the eye.

In some magnetic surgical apparatuses, the at least one opening comprises: a first opening disposed along a perimeter of the magnetic body; and a second opening disposed along the perimeter of the magnetic body and disposed opposite the first opening. In some embodiments, the first opening and the second opening are formed as loops coupled to the magnetic body. In some embodiments, the magnetic body comprises a diameter or length in a range of 1 mm to 20 mm.

Some magnetic surgical apparatus have a magnetic body that comprises a rare earth magnet or an electromagnet.

Other aspects concern magnetic surgical apparatuses, comprising: a magnetic body configured for internal placement within a patient body; and an attachment apparatus coupled to the magnetic body. In some embodiments, the magnetic body is configured for internal placement behind an eye of the patient. Certain magnetic bodies comprises an arcuate surface. In certain embodiments, the arcuate surface comprises a first arcuate line taken along a first direction of the accurate surface, and a second arcuate line taken along a second direction orthogonal to the first arcuate line to form a concave surface configured to mateably couple adjacent a rear surface of the eye.

Certain magnetic bodies comprise an arcuate surface in orthogonal directions for form a concave surface configured to mateably couple adjacent a rear surface of the eye. In come embodiments, the attachment apparatus comprises an arcuate radius of curvature substantially similar to the concave surface of the magnetic body.

Some attachment apparatuses comprise: an arm with a first end coupled to the magnetic body; and a handle coupled to a second end of the arm opposite the first end of the arm, the handle configured to position the magnetic body behind the eye.

In certain embodiments, the arm is releasably coupled to the magnetic body and configured to position the magnet behind the eye and then be released from the magnetic body and removed from behind the eye while the magnet remains behind the eye.

In certain embodiments, the arm is permanently coupled to the magnetic body and is configured to position the magnet behind the eye and then be removed with the magnetic body from behind the eye.

Certain attachment apparatuses comprises at least one opening disposed along a perimeter of the magnetic body, the at least one opening configured to receive sutures for coupling the magnetic body adjacent a rear portion of the eye. In some embodiments, the at least one opening comprises: a first opening disposed along a perimeter of the magnetic body; and a second opening disposed along the perimeter of the magnetic body and disposed opposite the first opening. In certain embodiments, the first opening and the second opening are formed as loops coupled to the magnetic body.

Some magnetic bodies comprises a diameter or length in a range of 5 mm to 20 mm. In certain embodiments, the magnetic body comprises an arcuate surface comprising an area in a range of 25 to 400 mm².

Some magnetic bodies comprises a rare earth magnet or an electromagnet.

In some embodiments, the magnetic body comprises an inert coating disposed around the magnet.

The foregoing and other aspects, features, and advantages will be apparent from the DESCRIPTION and DRAWINGS, and from the CLAIMS if any are included.

### BRIEF DESCRIPTION OF THE DRAWINGS

The drawings illustrates certain non-limiting embodiments and implementations.

FIG. 1A is a representation of a front side-view of a person or patient centered around the left of eye of the patient.

FIG. 1B is a cross-sectional side-view perpendicular to the view shown in FIG. 1A, and is taken along the section line 1B shown in FIG. 1A.

FIG. 2A is a representation of another cross-sectional side-view of the patient eye and eye socket similar to what was shown in FIG. 1B.

FIG. 2B is a perspective view of a patient eye shown without the context of the surrounding eye socket or other structures and tissue.

FIG. 3A shows a perspective view of the magnetic body.

FIG. 3B shows a perspective view of another non-limiting example of a magnetic body similar to the magnetic body of FIG. 3A.

FIG. 3C shows a top plan view of another non-limiting example of a magnetic body similar to the magnetic body of FIG. 3B.

FIG. 4A shows a cross-sectional side view of an entire eye with the lens on the left and the optic nerve on the right, and an enlarged cross-section view of a rear portion of the eye illustrating further detail of the macula and the fovea.

FIG. 4B further shows the magnetic surgical apparatus being placed within the patient and on a back surface or adjacent an area or tissue to be treated, such as the patient's eye.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices, and technologies to which the disclosed technologies may be applied. The full scope of the technology disclosed herein is not limited to the examples that are described below.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the disclosure that may be embodied in various forms. Therefore, specific structural and functional details disclosed herein are not to be interpreted as limits, but merely as a basis for teaching one skilled in the art to employ the present disclosure. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing certain embodiments by way of example only and is not intended to be limiting of the claimed inventions. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

This disclosure, its aspects and implementations, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

While this disclosure includes embodiments of many different forms, there is shown in the drawings and will herein be described in detail particular embodiments with the understanding that the present disclosure is to be considered as an exemplification of the principles of the disclosed methods and systems, and is not intended to limit the broad aspect of the disclosed concepts to the embodiments illustrated.

All amounts, ratios, and percentages are by weight unless otherwise indicated. The articles “a”, “an”, and “the” each refer to one or more, unless otherwise indicated by the context of the specification. Thus, for example, reference to “a step” includes reference to one or more of such steps.

The disclosure of ranges includes the range itself and also anything subsumed therein, as well as endpoints. For example, disclosure of a range of 2.0 to 4.0 includes not only the range of 2.0 to 4.0, but also 2.1, 2.3, 3.4, 3.5, and 4.0 individually, as well as any other number subsumed in the range. Furthermore, disclosure of a range of, for example, 2.0 to 4.0 includes the subsets of, for example, 2.1 to 3.5, 2.3 to 3.4, 2.6 to 3.7, and 3.8 to 4.0, as well as any other subset subsumed in the range. As used herein “about,” “approximately,” and “substantially” mean within a percent difference of less than or equal to 20%, 10%, 5%, 3%, 2%, or 1%. Similarly, the disclosure of Markush groups includes the entire group and also any individual members and subgroups subsumed therein.

This disclosure provides a system and apparatus for using a magnet disposed within a patient for the attraction and orientation of magnetic cells 200, including placement of the magnetic surgical apparatus 135 behind an eye 10 of a patient, such as adjacent a rear portion of the eye 10 of the patent. The placement of the magnet may be spatially separate and independent of the infusion of new cells, with temporal overlap to allow the positioned magnet and magnetic field 180 to orient and position the magnetic cells 200 in a desired location.

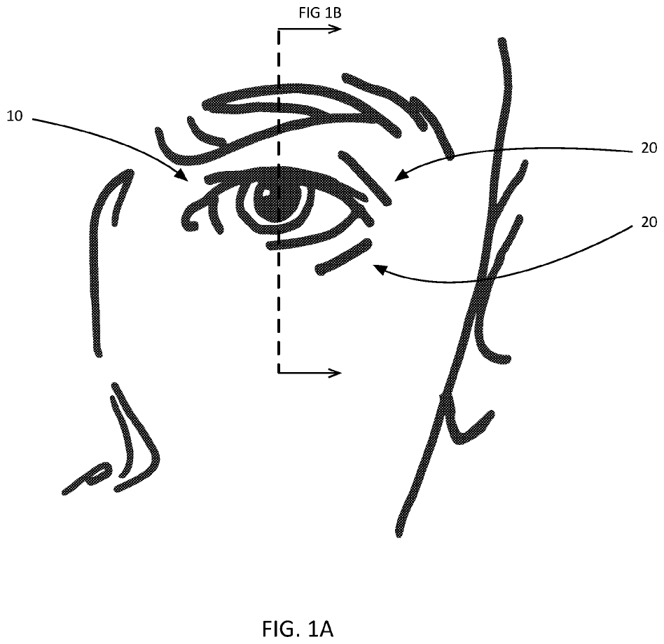

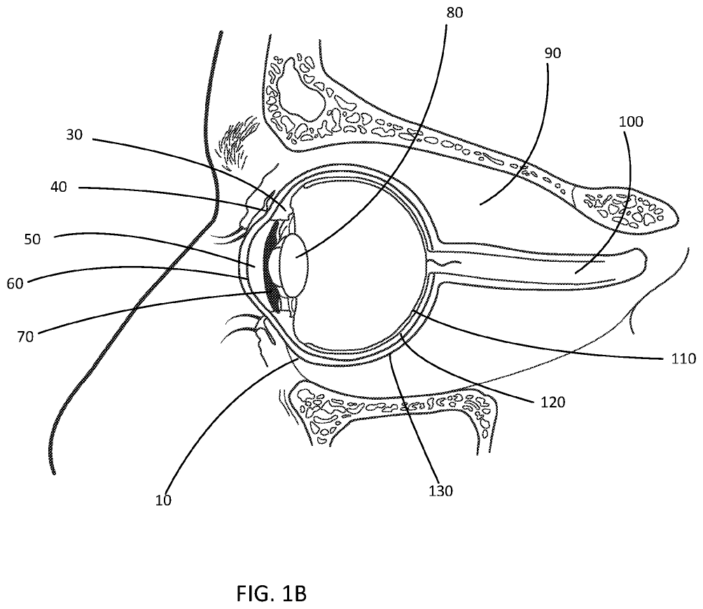

FIG. 1A, included below, is a front side-view of a person or patient centered around the left of eye 10 of the patient. While the patient illustrated in FIG. 1A is a human patient, other animal patients may also benefit from the ocular surgical magnet apparatus disclosed herein. FIG. 1A also shows incisions 20 may be made through the skin surrounding the eye 10, such as above or below an outer lateral portion of the eye 10, or in those locations underneath the eyelids entering through the conjunctiva. Other incision sites may be selected based on positioning of the ocular magnet apparatus and portion of the eye 10 to be treated.

FIG. 1B, included below, is a cross-sectional side-view perpendicular to the view shown in FIG. 1A, and is taken along the section line 1B shown in FIG. 1A. FIG. 1B shows additional features of the eye 10 including ciliary body 30, conjunctiva 40, anterior chamber 50, cornea 60, iris 70, lens 80, orbit 90, optic nerve 100, retina 110, Choroid 120 and sclera 130.

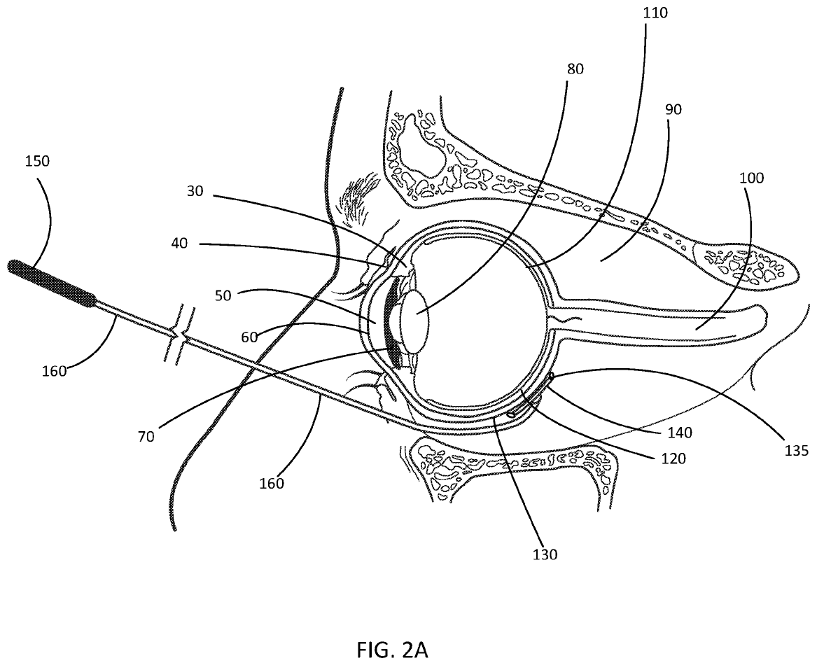

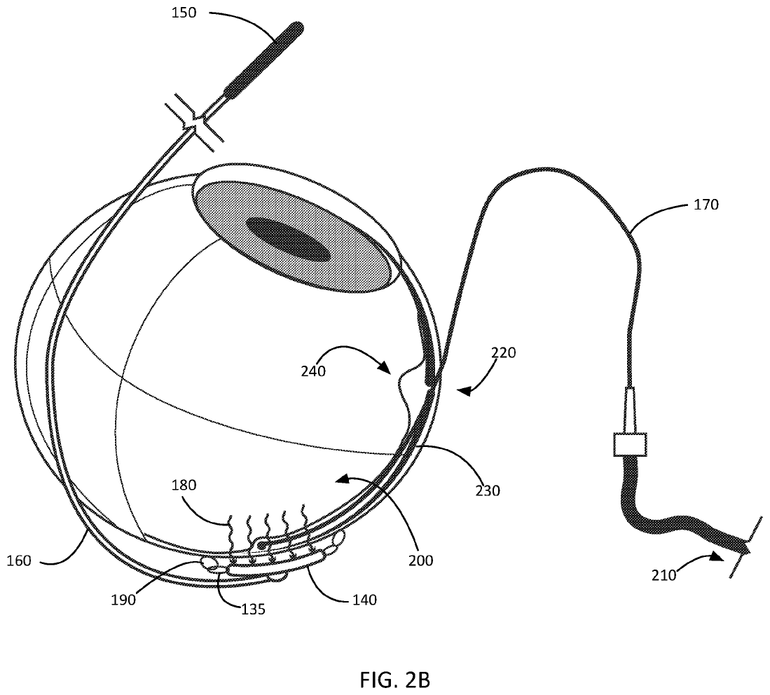

FIG. 2A, included below, is another cross-sectional side-view of the patient eye 10 and eye socket similar to what was shown in FIG. 1B. FIG. 2A differs from FIG. 1B by showing a magnetic surgical apparatus 130 positioning a magnetic body 140 behind the eye 10 of the patient, where magnets have not previously been placed. The magnetic body 140 is shown being placed by an arm 160 of the magnetic surgical apparatus 135, to which the magnetic body 140 is coupled, the arm 160 of the magnetic surgical apparatus 135 extending through the incision in the skin and around to a desired portion of the patient's eye 10. The arm 160 and the magnetic body 140 may be manipulated and positioned by the handle 150 coupled to the arm 160.

FIG. 2B is a perspective view of a patient eye 10 shown without the context of the surrounding eye socket or other structures and tissue. Like FIG. 2A, FIG. 2B shows (but with greater magnification) a magnetic surgical apparatus 135 positioning a magnetic body 140 behind or at a rear portion of the patient eye 10, such as over the macula or at an area where retinal cells have degraded, been damaged, or are otherwise not functioning properly and will be replaced or augmented with new magnetic stem cells 200. FIG. 2B further shows a catheter 170 that may be used to place new magnetic stem cells at or near a site of the damaged retina. In some embodiments, the catheter 170 has a connection to a pump 210. FIG. 2B also shows sclerotomy and choroidal fistula 220, subretinal cannulation 230, and Healon lifter retina 240. The magnetic body 140 may be positioned before the placement of the new magnetic cells 200 to assist in the positioning and alignment of the magnetic cells 200. As shown in FIG. 2B, the magnetic body 140 may create a magnetic field 180 that draws the magnetic cells 200 to a desired position. A size, shape, position, and orientation of the magnetic body 140 may be configured to match or approximate a size, shape, position, and orientation of the damaged portion of the eye 10 and the area in which new magnetic cells 200 will be positioned.

Accordingly, FIG. 2B shows the magnetic surgical apparatus 135, comprising a magnetic body 140. The magnetic body 140 comprises an arcuate surface and is configured for internal placement behind an eye 10 of a patient, the arcuate surface comprising an area in a range of 1 to 400 mm², or 25 to 400 mm², or in instances where tighter or smaller treatment areas such as 1 to 25 mm² are desired. The magnetic surgical apparatus 135 comprises an inert coating disposed around the magnet. The magnetic surgical apparatus 135 comprises an attachment apparatus comprising an arcuate radius of curvature substantially similar to a radius of curvature of the magnetic body 140. The attachment apparatus is coupled to the magnetic body 140 and further comprises at least one opening disposed along a perimeter of the magnetic body 140, the at least one opening configured to receive sutures 190 for coupling the magnetic body 140 adjacent a rear portion of the eye 10. The magnetic surgical apparatus 135 may comprise an arm 160 with a first end coupled to the magnetic body 140 and a second end of the arm 160 opposite the first end of the arm 160. The magnetic surgical apparatus 135 may comprise a handle 150 coupled to the second end of the arm 160, the handle 150 being configured to position the magnetic body 140 behind the eye 10 of the patient.

The arcuate surface of the magnetic body 140 may comprise a first arcuate line taken along a first direction of the accurate surface, and a second arcuate line taken along a second direction orthogonal to the first arcuate line to form a concave surface configured to mateably couple adjacent a rear surface of the eye 10. The magnetic body 140 may comprise arcuate surfaces in orthogonal directions to form a concave surface configured to mateably couple adjacent a rear surface of the eye 10. The arcuate radius of curvature of the attachment apparatus may be substantially similar to the arcuate radius of curvature of the magnetic body 140. The arm 160 may be releasably coupled to the magnetic body 140 and configured to position the magnet behind the eye 10 and then be released from the magnetic body 140 and removed from behind the eye 10 while the magnet remains behind the eye 10. The arm 160 may be permanently coupled to the magnetic body 140 and be configured to position the magnet behind the eye 10 and then be removed with the magnetic body 140 from behind the eye 10.

The at least one opening may comprise a first opening disposed along a perimeter of the magnetic body 140, and a second opening disposed along the perimeter of the magnetic body 140 and disposed opposite the first opening. The first opening and the second opening may be formed as loops coupled to the magnetic body 140. The magnetic body 140 may comprise a diameter or length in a range of 1 mm to 20 mm, 5 mm to 20 mm, or in instances of smaller treatment areas 1 mm to 5 mm. The magnetic body 140 may comprise a rare earth magnet or an electromagnet.

The fixed/rare earth magnets, electromagnets, or superconducting magnets may comprise sufficient field density uniformity and magnetic field gradient to direct the cells and hold them in place. Specifics of magnetic field strength may vary by need, such that stronger fields/gradients being used when the magnet is required to act at greater distances, across larger areas or surfaces of damaged cells, and weaker fields/gradients may be used when the magnet can be localized closer to the implanted particles and/or target tissues. The magnetic cells 200 may be directed to the target tissue and with a first magnetic field of a first strength, with the magnetic field 180 then (or thereabout) being modulated to further refine their movement and shape the tissue. The magnetic field 180 may be modulated to be a second magnetic field of a second strength less than the first strength to merely hold the cells in a desired location until a desired cell adhesion is achieved, rather than using the stronger first field to move the cells.

The magnetic field 180 may also be modulated by activating different zones, portions, or areas of the magnet or magnetic body 140 to encourage movement of migrations of the cells to particular areas. Cell migration may be directed in real time while observing the placement of cells, such as when cells include a visual marker and may be observed visually during placement. Cell placement may also be observed or monitored indirectly or in any other suitable way. In some instance, multiple layers or strata of cells may be placed with a delay between layering so as to provide time for adhesion of a first layer, and then a subsequent second (or any number or 1+n) layer of cells may be added. In some instances, a first layer and subsequent layer of cells may comprise a same or similar footprint, while in other instances a footprint of subsequent layers of cells may vary, and may include different cell types.

The magnetic body 140 may be disposed within the patient rather than having the magnet being externally applied, such as being affixed in a patch to the surface of the eye external to the eyelid centered over the cornea (so as to drive, push, or repel the magnetic cells 200, such as magnetic human corneal endothelial cells that are floating in the anterior chamber towards the endothelium). The magnetic body 140 will be disposed within the patient and proximate the area being treated, such as within the eye socket and behind the eye 10 of the patient (so as to attract or pull the magnetic cells 200 to a desired location on or underneath the neural retina in the eye 10).

The magnetic body 140 may be left in position for a desirable length of time, during which time the cells may begin to adhere, bond, attach, or grow into place. For example, the magnet may be left in place for 5 minutes to 7 days, and in some instances more typically 1 hour to 3 days. The magnetic field 180 would help affix the donor, nanoparticle-bound cells (such as photoreceptor cells or retinal pigment epithelial cells or stem cells) to the vitreal surface or subretinal Bruch's membrane surface of the host/patient, after which time natural cell adhesion would take place, removing the need for additional magnetic field 180 application. The internal magnet could be removed.

Several unadhered cells could be reduced with respect to applications where cells are not drawn and held into place, thereby reducing a number of unadhered cells that would otherwise die and remain free floating away or otherwise localize at a distance from the desired bonding site.

With time, the nanoparticles on the surface of the donor cells would degrade from the surfaces by natural proteolytic mechanisms and be washed away in the fluid of the anterior chamber. Similarly, with time, intracellular nanoparticles within the donor cells would be released from the cells by natural mechanisms and be washed away in the fluid of the anterior chamber. The small size of the magnetic particles would allow outflow through veins or through lymphatics or through the trabecular meshwork and other natural outflow pathways without clogging these pathways or elevating intraocular pressure. The delivery of the magnetic cells 200 to the internal retinal surfaces for retinal cell therapies would allow improved retinal function and removal of accompanying fluid from the cell injection. The retina would subsequently become more functional by virtue of preventing loss, replacing, or both preventing loss and replacing, retinal cells or retinal pigment epithelium or some combination of these cell types, thereby improving patient vision, reducing edema, and decreasing the pain typically associated with edematous tissue or organs.

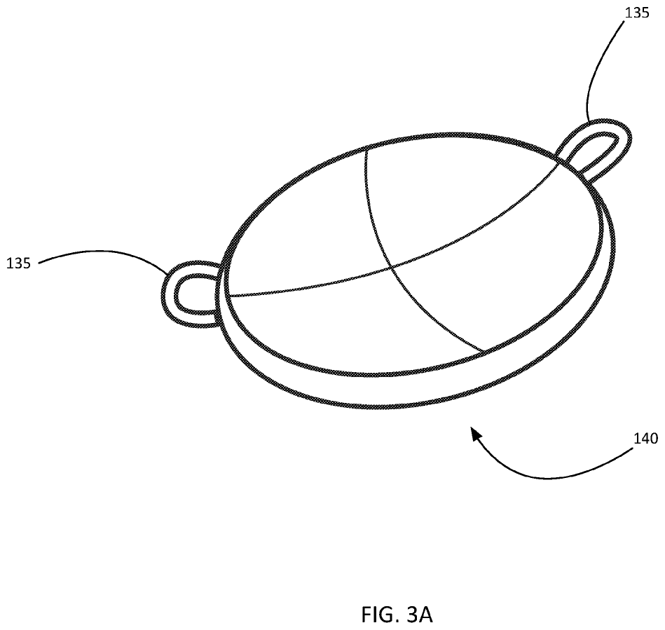

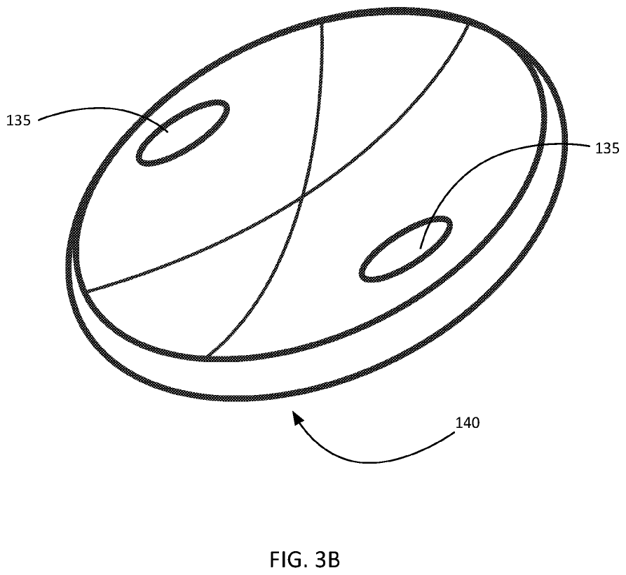

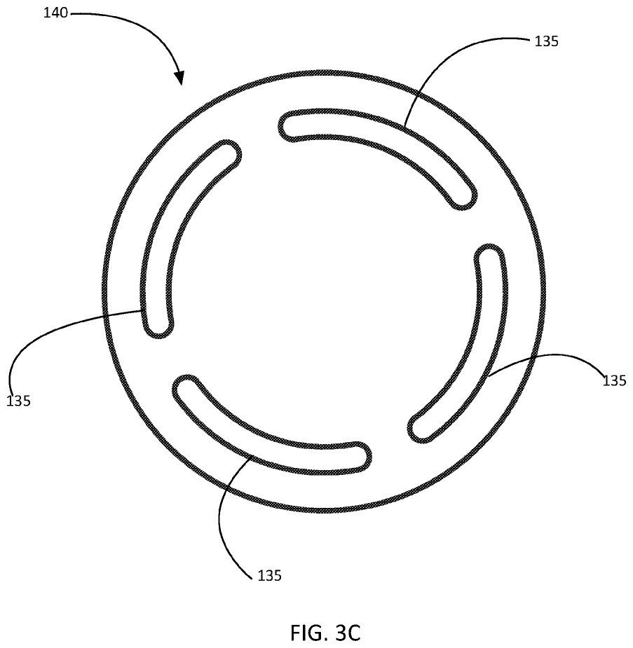

FIG. 3A, included below, shows a perspective view of the magnetic body 140, showing the concave surface or underside of the magnetic body 140 facing up or towards the viewer, the concave surface being sized so as to mateably couple with the outer curvature of the patient's eye 10. Where the magnetic body 140 is configured to mateably couple with other structures of tissue, the mateable surface of magnetic body 140 may be correspondingly adjusted. FIG. 3A also shows a number of attachment apparatuses 135 coupled to the magnetic body 140. There may be 2, 3, 4, dozens, 1-100, or any desirable number of attachment apparatus, including loops, rings, or openings configured to receive sutures of stitches for coupling the magnetic body 140 to the patient.

FIG. 3B, included below, shows a perspective view of another non-limiting example of a magnetic body 140 similar to the magnetic body 140 of FIG. 3A. The magnetic body 140 of FIG. 3B differs from that of FIG. 3A by showing the attachment apparatus 135 being formed as openings withing an outer perimeter of the magnetic body 140.

FIG. 3C, included below, shows a top plan view of another non-limiting example of a magnetic body 140 similar to the magnetic body 140 of FIG. 3B, which shows the attachment apparatus 135 formed as four elongated openings dispose around, and offset within, the outer perimeter of the magnetic body 140, although any desired number of openings may be present.

FIG. 3B, included below, shows a perspective view of another non-limiting example of a magnetic body 140 similar to the magnetic body 140 of FIG. 3A. The magnetic body 140 of FIG. 3B differs from that of FIG. 3A by showing the attachment apparatus 135 being formed as openings withing an outer perimeter of the magnetic body 140.

FIG. 3C, included below, shows a top plan view of another non-limiting example of a magnetic body 140 similar to the magnetic body 140 of FIG. 3B, which shows the attachment apparatus 135 formed as four elongated openings dispose around, and offset within, the outer perimeter of the magnetic body 140, although any desired number of openings may be present.

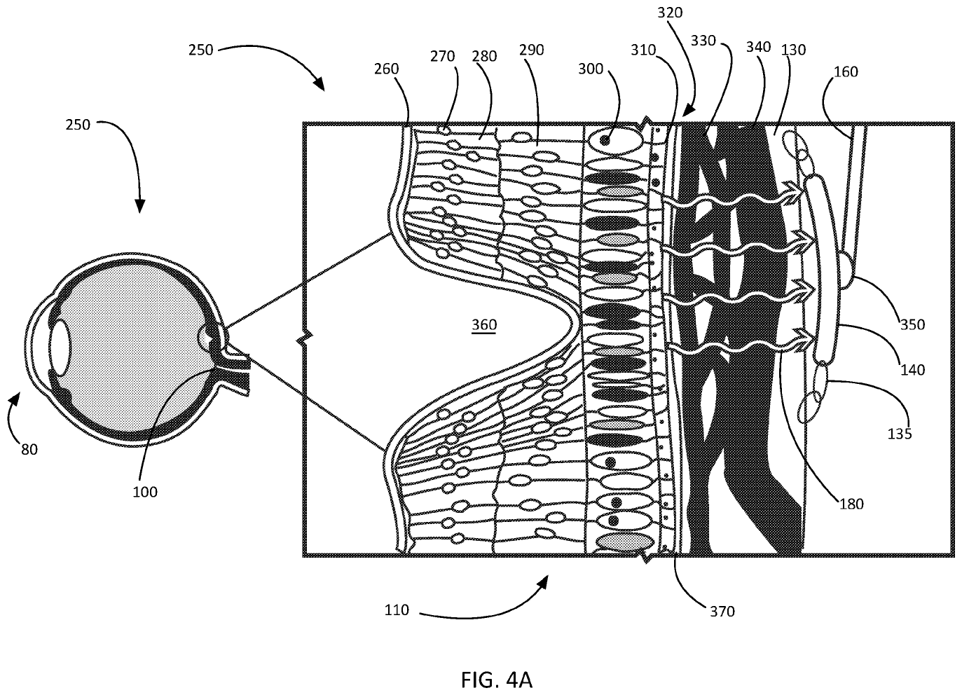

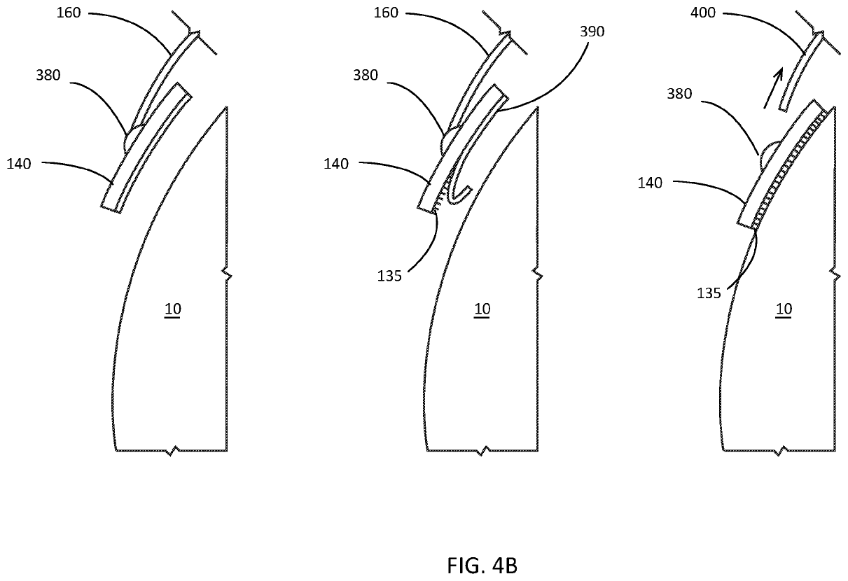

FIG. 4A, included below, shows a cross-sectional side view 250 of an entire eye 10 with the lens 80 on the left and the optic nerve 100 on the right, and an enlarged cross-section view of a rear portion of the eye 10 illustrating further detail of the macula and the fovea 360. FIG. 4A further shows the magnetic surgical apparatus 135 coupled on the rear of the sclera 130 (not shown to scale), behind the retina 110 to provide a magnetic field 180 to direct the placement of the newly inserted magnetic cells 200, such as RPE cells or photoreceptor cells or retinal ganglion cells or stem cells.

FIG. 4A further shows the magnetic surgical apparatus 135 positioned within the patient and behind the eye 10, to attract new cells to a desired location within the eye 10, such as near or behind the fovea 360. In other instances, the magnetic surgical apparatus 135 may be positioned at the retina 110, or at or near the macula and away (or laterally offset) from the fovea 360 so as to avoid introducing new cells (such as by a catheter 170 or cannula) in such a way—or at a sensitive location—where a patient's sight and vision may be damaged. In either event, placement may be determined according to patient need, desired patient outcome, and acceptable risk. In some instances, and without limitation, the magnetic surgical apparatus 135 may be positioned at the retina 110, or at or near the macula and away (or laterally offset) from the fovea 360 so that the patient's sight may be improved without a risk of inserting cells near the fovea 360 or other sensitive or delicate structure that may be potentially damaged, such as the fovea 360.

Additional features shown in FIG. 4A include nerve cells 260, ganglions 270, inner plexiform layer 280, inner nuclear layer 290, photo receptors 300, retinal pigment epithelium (RPE) 310, new cells introduced 320, capillaries 330, chroid 340, optical release 350, and Bruch's membrane 370.

FIG. 4B, included below, further shows the magnetic surgical apparatus 135 being placed within the patient and on a back surface or adjacent an area or tissue to be treated, such as the patient's eye 10. The magnetic surgical apparatus 135 may be positioned by manipulating the attached arm 160, which may then be subsequently removed or detached after placement (release 380). After removal of the arm 160 (yielding the detached arm 400), the magnetic body 140 may remain until desired adhesion of the new magnetic cells 200 is achieved. After desired adhesion is achieved the magnetic body 140 may remain in situ, or alternatively, may be removed, such as by reattaching the arm 160, disengaging the attachment apparatus 135, and removing the magnetic body 140. In instances where the magnet is small and remain, the remaining magnet may be a battery powered electromagnet that will run out of battery and cease to generate a magnetic field 180 or operate as a magnet after a desired period of time.

The attachment apparatus 135 may comprise any suitable form of attachment, including one or more of: (i) openings, rings, or loops for sutures, (ii) an adhesive, and (iii) retractable teeth, tines, barbs, hook fasteners, or claws that may be housed within the magnetic body 140 and may be activated with or through the arms 160, such as by mechanical movement (twisting or engaging a button or lever) or by electromechanical movement. When the attachment apparatus 135 of choice comprises an adhesive, the adhesive may be covered by a peel-way surface 390 of backing or inert material that can protect the adhesive until the magnetic body 140 is near the treatment site. The backing may thus protect the adhesive during positioning of the magnetic body 140 and then may be removed with a removal device, such as a line or string, In other instances, the adhesive may be uncovered, but not bond to the treatment site until being positioned and activated by moisture, temperature, a magnetic field 180, or other suitable stimulus, such as a stimulus delivered by the magnetic surgical apparatus 135, from outside the magnetic surgical apparatus 135, or by a substance at, proximate, or adjacent the treatment site.

It will be understood that the embodiments disclosed are not limited to the specific components disclosed herein, as virtually any components consistent with the intended operation of a method and/or system implementation for such an embodiment may be utilized. Accordingly, for example, although particular component examples may be disclosed, such components may be comprised of any shape, size, style, type, model, version, class, grade, measurement, concentration, material, weight, quantity, and/or the like consistent with the intended purpose, method and/or system of implementation. In places where the description above refers to certain implementations or embodiments, it should be readily apparent that a number of modifications may be made without departing from the scope and/or spirit thereof and that these principles and modifications may be applied to other such embodiments. The presently disclosed embodiments are, therefore, to be considered in all respects as illustrative and not restrictive.

Many additional material types, components, methods, and procedures known in the art are contemplated for use with certain implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

Many additional implementations are possible. Further implementations are within the claims.

1. A magnetic surgical apparatus, comprising:
a magnetic body comprising an arcuate surface and configured for internal placement behind an eye of a patient, the arcuate surface comprising an area in a range of 1 to 400 mm²;
an inert coating disposed around the magnet;
an attachment apparatus comprising an arcuate radius of curvature substantially similar to a radius of curvature of the magnetic body, the attachment apparatus coupled to the magnetic body and further comprising at least one opening disposed along a perimeter of the magnetic body, the at least one opening configured to receive sutures for coupling the magnetic body adjacent a rear portion of the eye;
an arm with a first end coupled to the magnetic body and a second end of the arm opposite the first end of the arm; and
a handle coupled to the second end of the arm, the handle configured to position the magnetic body behind the eye of the patient.

2. The magnetic surgical apparatus of claim 1, wherein the arcuate surface comprises a first arcuate line taken along a first direction of the accurate surface, and a second arcuate line taken along a second direction orthogonal to the first arcuate line to form a concave surface configured to mateably couple adjacent a rear surface of the eye.

3. The magnetic surgical apparatus of claim 1, wherein the magnetic body comprises arcuate surfaces in orthogonal directions to form a concave surface configured to mateably couple adjacent a rear surface of the eye.

4. The magnetic surgical apparatus of claim 1, wherein the arcuate radius of curvature of the attachment apparatus is substantially similar to the arcuate radius of curvature of the magnetic body.

5. The magnetic surgical apparatus of claim 1, wherein the arm is releasably coupled to the magnetic body and configured to position the magnet behind the eye and then be released from the magnetic body and removed from behind the eye while the magnet remains behind the eye.

6. The magnetic surgical apparatus of claim 1, wherein the arm is permanently coupled to the magnetic body and is configured to position the magnet behind the eye and then be removed with the magnetic body from behind the eye.

7. The magnetic surgical apparatus of claim 1, wherein the at least one opening comprises:
a first opening disposed along a perimeter of the magnetic body; and
a second opening disposed along the perimeter of the magnetic body and disposed opposite the first opening.

8. The magnetic surgical apparatus of claim 7, wherein the first opening and the second opening are formed as loops coupled to the magnetic body.

9. The magnetic surgical apparatus of claim 1, wherein the magnetic body comprises a diameter or length in a range of 1 mm to 20 mm and/or the magnetic body comprises a rare earth magnet or an electromagnet.

10. (canceled)

11. A magnetic surgical apparatus, comprising:
a magnetic body configured for internal placement within a patient body; and
an attachment apparatus coupled to the magnetic body.

12. The magnetic surgical apparatus of claim 11, wherein the magnetic body is configured for internal placement behind an eye of the patient.

13. The magnetic surgical apparatus of claim 11, wherein the magnetic body comprises an arcuate surface.

14. The magnetic surgical apparatus of claim 1, wherein the arcuate surface comprises a first arcuate line taken along a first direction of the accurate surface, and a second arcuate line taken along a second direction orthogonal to the first arcuate line to form a concave surface configured to mateably couple adjacent a rear surface of the eye.

15. The magnetic surgical apparatus of claim 11, wherein the magnetic body comprises an arcuate surface in orthogonal directions for form a concave surface configured to mateably couple adjacent a rear surface of the eye and/or the attachment apparatus comprises an arcuate radius of curvature substantially similar to the concave surface of the magnetic body.

16. (canceled)

17. The magnetic surgical apparatus of claim 11, wherein the attachment apparatus comprises:
an arm with a first end coupled to the magnetic body; and
a handle coupled to a second end of the arm opposite the first end of the arm, the handle configured to position the magnetic body behind the eye.

18. The magnetic surgical apparatus of claim 17, wherein the arm is releasably coupled to the magnetic body and configured to position the magnet behind the eye and then be released from the magnetic body and removed from behind the eye while the magnet remains behind the eye.

19. The magnetic surgical apparatus of claim 17, wherein the arm is permanently coupled to the magnetic body and is configured to position the magnet behind the eye and then be removed with the magnetic body from behind the eye.

20. The magnetic surgical apparatus of claim 18, wherein the attachment apparatus comprises at least one opening disposed along a perimeter of the magnetic body, the at least one opening configured to receive sutures for coupling the magnetic body adjacent a rear portion of the eye.

21. The magnetic surgical apparatus of claim 19, wherein the at least one opening comprises:
a first opening disposed along a perimeter of the magnetic body; and
a second opening disposed along the perimeter of the magnetic body and disposed opposite the first opening and optionally, the first opening and the second opening are formed as loops coupled to the magnetic body.

22. (canceled)

23. The magnetic surgical apparatus of claim 11, wherein the magnetic body comprises a diameter or length in a range of 5 mm to 20 mm; the magnetic body comprises an arcuate surface comprising an area in a range of 25 to 400 mm; the magnetic body comprises a rare earth magnet or an electromagnet; and/or the magnetic body comprises an inert coating disposed around the magnet.

24-26. (canceled)
